SEQUENCE LISTING (4 sequences)

SEQ ID NO: 1 - protein, 521 aa, Artificial Sequence
  Fusion protein containing a Human TWEAK Receptor Binding Domain, a Pseudomonas Exotoxin mutant protein, and a synthetic sequence
MAHHHHHHVGTGSNDDDKSPDLARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVHGGGGSPEGGSLAALTAHQACHLPLETFTLHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 2 - protein, 521 aa, Artificial Sequence
  Fusion protein containing a Pseudomonas exotoxin mutant protein, a Human TWEAK Receptor Binding Domain, and a synthetic sequence
MAHHHHHHVGTGSNDDDKSPDLPEGGSLAALTAHQACHLPLETFTLHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLKGGGGSARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 3 - protein, 33 aa, Artificial Sequence
  Synthetic trimerization domain Isoleucine zipper (From BBRC 321, 930 (2004))
RMKQIEDKIEEILSKIYHIENEIARIKKLIGER

SEQ ID NO: 4 - protein, 149 aa, Homo sapiens
TRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

(19) United States
(12) Patent Application Publication
(10) Pub. No.: US 2008/0175896 A1
(43) Pub. Date: Jul. 24, 2008

(54) Tweak-pseudomonas exotoxin a fusion protein for cancer therapy

(76) Inventors: Jeffrey A. Winkles, Frederick, MD (US); Sharron Brown, Gaithersburg, MD (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 11/998,518

(22) Filed: Nov. 30, 2007

Related U.S. Application Data
(60) Provisional application No. 60/872,359, filed on Dec. 1, 2006.

(51) Int. Cl. A61K 9/127; C07K 19/00; C07K 14/21; C12N 5/06; A61K 38/16

(52) U.S. Cl. 424/450; 530/300; 530/402; 435/375; 514/12; 530/391.7

(57) ABSTRACT

Soluble TNF superfamily ligand monomers, including TWEAK, spontaneously associate into a homotrimeric structure that binds with high affinity to a cell surface receptor(s). A TWEAK is fused with a modified (mutant) Pseudomonas exotoxin (PE38). This fusion protein's TWEAK domain binds with high affinity to a fibroblast growth factor-inducible 14 (Fn14) cell surface receptor. These fusion proteins act as cytotoxins targeting various cells, diseased cells, such as cancer cells, and cells undergoing cellular insult response.

### CROSS REFERENCE TO RELATED APPLICATIONS

The present application claims priority under 35 U.S.C. §119 to the U.S. Provisional Patent Application Ser. No. 60/872,359, filed on Dec. 1, 2006, which is herein incorporated by reference in its entirety.

### STATEMENT REGARDING FEDERALLY SPONSORED RESEARCH OR DEVELOPMENT

The U.S. Government has certain rights in this invention. The U.S. Government has a paid-up license in this invention and the right in limited circumstances to require the patent owner to license others on reasonable terms as provided for by the terms of Grant Nos. HL39727 and NS55126 awarded by NIH. This work was also supported, in whole or in part, by funding from the Susan G. Komen Breast Cancer Foundation.

### FIELD OF THE INVENTION

The present invention generally relates to the field of oncology, and particularly to the creation and use of fusion proteins targeting specific cell-surface antigens or receptors allowing the binding of these fusion proteins and their toxin moeity with malignant cells.

### BACKGROUND OF THE INVENTION

Traditional brain cancer therapies such as surgery, chemotherapy and radiation have a marginal impact on patient survival (1,2,4,5). Targeted therapy directed at specific growth factors, angiogenic factors, cell surface receptors, or intracellular signaling molecules implicated in glioma pathogenesis may provide an effective alternative (or combination) treatment strategy (1,2,4,5). A number of targeted therapies are under development for treatment of malignant glioma and several clinical trials are presently underway (1,2,4). One type of cell surface-targeted therapy under development is targeted toxin therapy. Targeted toxins consist of either a tumor cell-selective antibody linked to a protein toxin (referred to as an immunotoxin) or a growth factor/cytokine fused to a protein toxin (referred to as a fusion protein toxin) (6-7). These recombinant chimeric proteins bind to a tumor cell-specific receptor, and this delivers the toxin into the cell. The toxins themselves must be modified to remove their cell-binding domain(s) or of course this targeting strategy will not work. Numerous plant or bacterial toxins have been used for targeted toxin therapy but Pseudomonas exotoxin (PE) and diphtheria toxin are two of the most commonly used toxins (7-8). Both of these bacterial toxins promote ADP ribosylation of elongation factor (EF)-2, which inhibits protein synthesis and causes cell death (8).

Several cell surface receptors have been identified that are expressed at low levels in normal brain tissue but overexpressed in malignant glioma tissue and in some cases fusion protein toxins have been engineered that bind these receptors. For example, biologically active fusion protein toxins targeted to the transferrin receptor (9), uPA receptor (10), EGFR (11-12), IL-4 receptor (13-14), and IL-13 receptor (15-17) have been successfully produced. Clinical trials have been conducted with all of these agents (usually the fusion proteins are delivered directly to the tumor cells by convection enhanced delivery) and benefits have been observed in some patients (4,6,8,9,12,13,16,18). Nevertheless, there is still a need to develop fusion protein toxins targeting other cell surface receptors, because there is heterogeneity in receptor expression among brain tumors and new targeted toxins may have a superior safety and activity profile.

### SUMMARY OF INVENTION

Accordingly the present invention provides a fusion protein that specifically targets cell-surface antigens or receptors found on diseased cells, such as cancerous cells, by using exemplary protein structures employing a TNF-related cytokine. In an exemplary embodiment of the present invention a cytotoxin (fusion protein) includes the TNF-like weak inducer of apoptosis (TWEAK) receptor binding domain fused with a Pseudomonas exotoxin (PE) mutant protein. In certain embodiments, the PE mutant has a protein sequence including amino acids 253 to 364 and 396 to 613, and missing certain amino acid sequences as will be described below, and may be referred to herein as PE38. It may be that these amino acid sequences may or may not be operably linked in the fusion protein of the current invention. In another exemplary embodiment of the current invention the TWEAK-PE fusion protein is additionally fused with an isoleucine zipper (IZ) which may increase biological activity.

In another exemplary embodiment, a cytotoxin includes a TWEAK receptor binding domain fused with a PE, wherein the TWEAK is positioned at either the amino or carboxyl terminus of the cytotoxin. In an exemplary preferred embodiment, the TWEAK receptor binding domain is positioned at the amino terminus of the cytotoxin. In certain embodiments of the current invention a serine linker is included to fuse the TWEAK and PE proteins to form the cytoxin. It is contemplated that various other “linkers” may be employed in forming the cytotoxin of the current invention.

The exemplary fusion protein may be employed in a method of inducing death in diseased cells. The fusion protein contains a toxin moiety which may promote cell death once internalized within the cytosolic compartment of a cell. The method of inducing death of a cell includes contacting the diseased cell with a cytotoxin comprising a tumor necrosis factor like weak inducer of apoptosis (TWEAK) receptor binding domain fused with a Pseudomonas exotoxin (PE38). As a result of this contact, the fusion protein, via the TWEAK receptor binding domain, binds an Fn14 cell surface receptor expressed on the cell. The expressed Fn14 cell surface receptor may, in certain exemplary preferred embodiments, be over-expressed on the cell surface where the cell is malignant (cancer of any type), diseased (e.g., artherosclerosis, stroke, rheumatoid arthritis), or undergoing some form of cellular response, such as regenerative and inflammation responses due to cellular insult. The binding of the fusion protein induces death of the diseased cell as a result of the apoptotic activity induced by the PE protein component of the cytotoxin.

An exemplary embodiment of the present invention may provide the fusion protein in various formulations, such as a composition of matter (ingestible or topical) and a solution (injectable). These various formulations may provide an embodiment of the current invention wherein the fusion protein may be used as a therapeutic. The therapeutic may be targeted for binding with a cancerous cell by the binding of the fusion protein with an Fn14-receptor site which may be overexpressed on Fn14 positive cells. In a further exemplary embodiment of the present invention, a method of treating a subject in need thereof is provided. The method includes the step of administering an effective amount of the cytotoxin of the current invention including a tumor necrosis factor like weak inducer of apoptosis (TWEAK) receptor-binding domain fused with a Pseudomonas exotoxin (PE38), wherein the TWEAK receptor-binding domain binds with a cell surface receptor expressed on the surface of a cell.

It is to be understood that both the foregoing general description and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1. An illustration representing the TWEAK-Fn14 axis. Most cells express two TWEAK isoforms, a membrane-bound form (mTWEAK) and a soluble form (sTWEAK). TWEAK, like other TNF superfamily members, contains an extracellular TNF homology domain (rectangle) and associates into non-covalently linked homotrimers. Trimeric TWEAK binds to Fn14 monomers containing a single cysteine-rich domain (oval), which promotes receptor trimerization and signal transduction (arrows). TWEAK produced by one cell is acting on a neighboring cell via both a paracrine pathway (sTWEAK) and a juxtacrine pathway (mTWEAK). TWEAK juxtacrine activity may vary as contemplated by those of ordinary skill in the art. TWEAK may function as an autocrine factor (i.e., the TWEAK produced by a cell binds the Fn14 receptors on that same cell). Abbreviations: Cyto, cytoplasmic compartment; PM, plasma membrane.

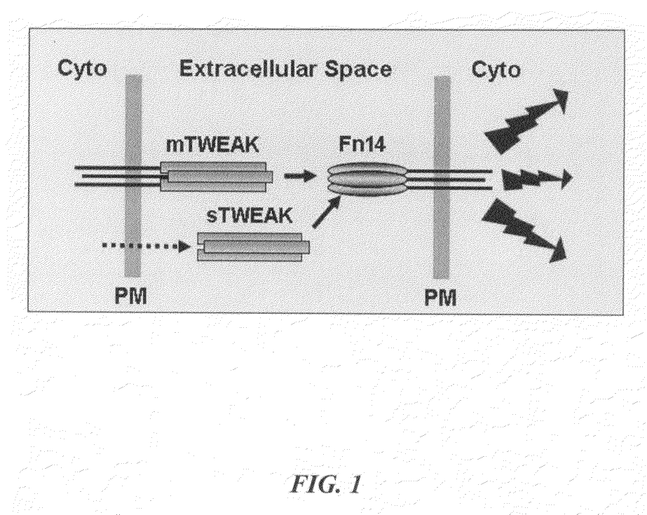

FIG. 2. A representation of TWEAK internalization and degradation following binding to Fn14-positive endothelial cells. Myc-tagged soluble TWEAK was purified from a stably-transfected HEK293 cell line and added (100 ng/ml) to human endothelial cell cultures at 4° C. for 30 min. Unbound myc-TWEAK was washed away and the cells were warmed to 37° C. to allow ligand internalization. The cells were harvested at the indicated times by trypsinization and equivalent amounts of protein were subjected to SDS-PAGE and Western blot analysis using anti-myc and anti-actin antibodies.

FIG. 3. A representation of TWEAK internalization following binding to Fn14-positive glioma cells. Serum-starved human U118 glioma cells plated on coverslips were either left untreated (0 min) or treated (100 ng/ml) with FLAG-tagged soluble TWEAK (Alexis Inc.). Unbound FLAG-TWEAK was washed away and 30 min later the cells were fixed in 3% buffered formalin. Indirect immunofluorescence analysis was performed using an anti-FLAG antibody (Sigma) or control mouse IgG (eBioscience) followed by incubation with a goat anti-mouse IgG F(ab′)2 Alexa Fluor 488 secondary antibody conjugate (Molecular Probes). Photographs were taken using a fluorescent microscope. Magnification is 100×.

FIGS. 4A, 4B, and 4C. Multiple diagrams providing an illustrative representation of the purified TWEAK-PE38, PE38-TWEAK, and PE38 protein constructs and evidencing their purification. (FIG. 4A) A schematic depiction of the TWEAK-PE38 (T-P), PE38-TWEAK (P-T) and PE38 (P) proteins is shown. The number of amino acids within each domain is provided underneath the T-P drawing. His=polyhistidine tag; G₄S=glycine-serine linker. A unique enterokinase cleavage site is also indicated. (FIGS. 4B and 4C). The three recombinant proteins were purified from bacterial cell extracts and equivalent amounts of protein were subjected to SDS-PAGE followed by either Coomassie blue staining (panel B, with MW standards on the far left) or Western blot analysis using an anti-PE antibody (panel C).

FIG. 5. A graphical representation evidencing the TWEAK-PE38 protein binding to the Fn14 receptor. Surface plasmon resonance analysis of TWEAK-PE38, PE38-TWEAK and PE38 binding to immobilized Fn14 was conducted. Sensograms showing real-time binding of each recombinant protein to Fn14-Fc immobilized on a CM5 chip is presented. A flow cell without any immobilized protein was used as the control for non-specific binding and these values were subtracted from the binding data.

FIG. 6. A graphical representation evidencing the TWEAK-PE38 protein as a cytotoxic agent. Breast cancer cells were plated in quadruplicate in a 96-well plate at a density of 2.5×10³ cells/well. The cells were either left untreated or treated with various doses of the recombinant TWEAK-PE38, PE38-TWEAK and PE38 proteins, as well as one concentration of recombinant human TWEAK (600 ng/ml). Cells were incubated for 48 hours at 37° C. and 5% CO₂. Cell survival, as estimated by DNA content/well, was measured using the CyQUANT cell proliferation assay kit (Invitrogen) according to the manufacturer's instructions.

FIG. 7. A block diagram illustrating a method of inducing death in a diseased cell through contacting the diseased cell with the cytotoxin of the current invention.

FIG. 8. A block diagram illustrating a method of treating a subject by administering an effective amount of the cytotoxin of the current invention.

### DETAILED DESCRIPTION OF THE INVENTION

Reference will now be made in detail to the presently preferred embodiments of the invention, examples of which are illustrated in the accompanying drawings.

Soluble tumor necrosis factor (TNF) superfamily ligand monomers, including TWEAK, spontaneously associate into a homotrimeric structure that binds with high affinity to an appropriate cell surface receptor(s). The TWEAK receptor is named Fn14.

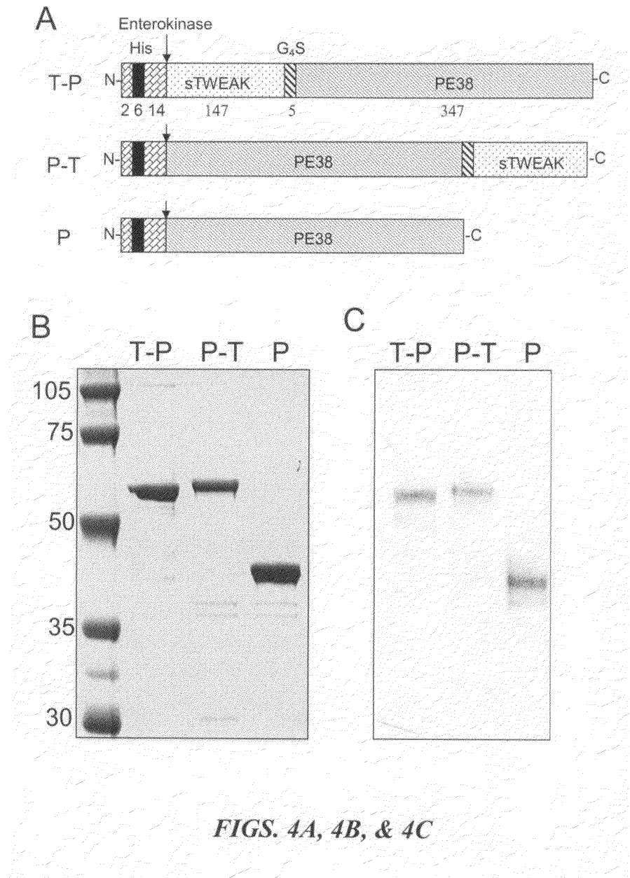

FIG. 4A shows two preferred embodiments of TWEAK-PE (Pseudomonas exotoxin A) fusion proteins which may be constructed by the current invention wherein the TWEAK receptor-binding domain is at the N-terminal region and the PE mutant called PE38 (active, toxic moiety) is at the C-terminal region (SEQ ID NO. 1) or, alternatively, wherein PE38 is at the N-terminal region and TWEAK is at the C-terminal region (SEQ ID NO. 2). FIG. 4A also shows PE38 alone, which may be used as negative control in pre-clinical assays. In a preferred embodiment, the TWEAK receptor-binding domain (SEQ ID NO. 4) is provided at the N-terminal region of the cytotoxin. Alternatively, the TWEAK receptor-binding domain may be provided at any position within the cytotoxin (fusion protein) and may include various other amino acids in various sequences that provide the benefit of binding with a high affinity to the Fn14 receptor. It is contemplated that the fusion proteins of the current invention may be combined with various other protein domains which may enhance the cytotoxic effect or increase the biological stabilty of the fusion protein. For example, the fusion proteins shown in FIGS. 4A and 4B may be engineered to contain an isoleucine zipper (IZ) trimerization domain (SEQ ID NO. 3).

It is contemplated that plasmids may be constructed and introduced into bacterial cells or insect cells for protein production purposes. For example, for bacterial cell production the pET Procaryotic Expression System (Novagen) may be used and many different plasmids and host strains may be utilized. For instance, the current invention may employ the plasmid pET45 (T7 promoter-based; IPTG-inducible; ampicillin resistance marker) and the genetically-engineered E. coli strain BL21(DE3)pLysS. This host strain has been successfully utilized for high-yield production of biologically active IL-13-PE38 fusion protein (31). DNA fragments encoding the TWEAK receptor-binding domain and the PE38 protein may be cloned into pET45 using PCR-based cloning strategies and appropriate restriction sites. The final constructs may be verified by DNA sequence analysis. Bacterial transformation may be performed as is known by those of ordinary skill in the art (31). Further, alternative techniques may be employed to construct and express the fusion proteins as contemplated by those of ordinary skill in the art. In particular, the DNA fragments may be cloned in baculovirus expression plasmids and expressed in insect cells. Alternatively, the expression of the fusion proteins may occur through use of mammalian, yeast, and/or plant cells. The contemplated use of various cell types and environmental conditions may all result in the expression of the fusion protein using the techniques and methodologies that are standard and well known to those of ordinary skill in the art for the cell types.

PE is a single-chain bacterial protein with three major domains: an N-terminal cell-binding domain (aa 1-252), an amphipathic helix-containing translocation domain containing a critical furin cleavage site (aa 253-364), and a C-terminal ADP-ribosylation domain (aa 396-613). In a preferred embodiment, the PE38 protein that is fused with the TWEAK is missing the entire cell-binding domain (aa 1-252) and domain 1b (aa 365-395). Further, in this embodiment, TWEAK fuses with the PE38 in the N-terminal location. Therefore, the TWEAK provides the binding affinity to the proper receptor for the fusion protein. In a further embodiment, and as shown in FIG. 4B, the TWEAK protein may be fused at the carboxyl terminus of the PE38 protein.

The proper receptor for the fusion proteins of the present invention may be the amino acid (aa) sequence of the fibroblast growth factor-inducible 14 (Fn14) receptor. The TWEAK-Fn14 ligand-receptor pair may play an important role in a variety of cellular processes (e.g, regenerative and/or inflammation response/cascade) and in the pathogenesis of several human diseases, including but not limited to atherosclerosis, stroke, rheumatoid arthritis, systemic lupus, nephritis, multiple sclerosis and other cellular insults from various diseases as may be contemplate by those skilled in the art. Further, because the Fn14 receptor is overexpressed in several types of cancers, such as liver, brain, breast, colon, cervical, testicular, and esophageal, whether malignant or benign, the current invention contemplates employing the TWEAK fusion protein against these and other types of cancer. It is further contemplated that the current invention may be employed against the various types of cells identified above, where those cells are in various stages of their disease progression or insult response. Thus, the cytotoxic effects of the fusion protein may be used against cells that are expressing (over-expressing) the Fn14 receptor, but that may not, in a clinical sense, have been fully transformed into “diseased” cells.

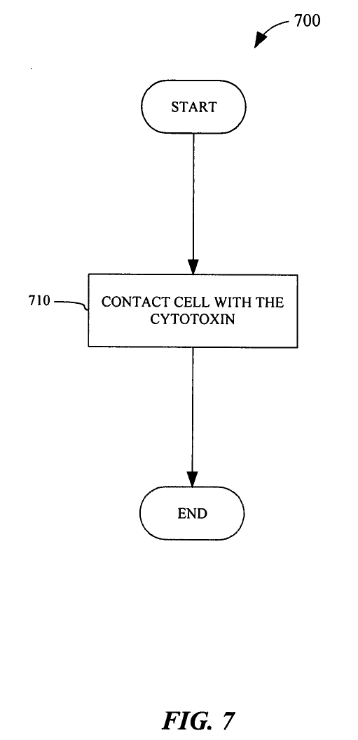

In a preferred embodiment, the TWEAK fusion protein is employed as a cytotoxin. The affinity of the TWEAK fusion protein to the Fn14 receptor may allow the TWEAK fusion protein to bind to malignant cells, various types of diseased cells, and other cells undergoing various processes, such as an inflammation response, and provide for the death of those cells. The present invention provides a method 700 for inducing cell death in a diseased cell. As shown in FIG. 7, method 700 includes a single step 710 of contacting a cell, such as a malignant or benign cancer cell, arthritic cell, ischemic cell, atherosclerotic cell, cell undergoing an inflammation response, pre-disease state cell expressing Fn14 receptor, and the like, with the one or more forms of the TWEAK fusion protein.

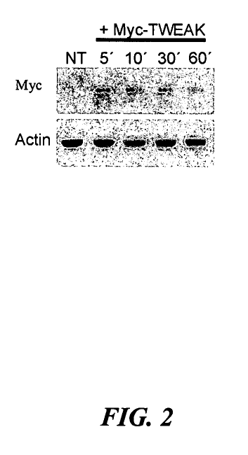

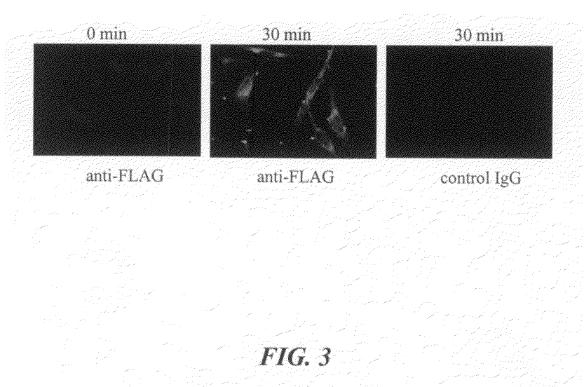

For example, the TWEAK fusion protein may be delivered to cancerous tumor cells, wherein the TWEAK fusion protein may contact, and from its binding affinity selectively bind with, the Fn14 positive cells. The binding of TWEAK fusion protein with the Fn14 receptor positions the TWEAK fusion protein on the cell surface and makes it available for internalization within the cell. Internalization may be promoted through the binding of various other components (e.g., catalysts, chaperones, assembly or transport biomolecules or the like) with the fusion protein to promote the internalization of the fusion protein of the current invention. Internalization of the TWEAK fusion proteins allows the toxin moiety to enter the cytosolic compartment of the cell. FIGS. 2 and 3 provide evidence for the efficient internalization of TWEAK by Fn14-positive cells. In a preferred embodiment, the delivery of the cytotoxin into the cell may occur through cleavage of the fusion protein in the Golgi-ER compartment and then the toxin itself translocating into the cytoplasm. Once in the cytoplasm the toxin may promote ADP ribosylation of elongation factor-2. This ribosylation may promote the inhibition of protein synthesis by the cell and cause cell death. It is contemplated that other intra-cellular mechanisms may be activated by the TWEAK fusion protein which may lead to cell death.

As described previously, the overexpression of the Fn14 surface receptor is predominant in various forms and types of cancerous cells, other diseased cells, and cells that may be undergoing an inflammation response (39). The TWEAK fusion protein may be bound with various identifiers, such as the Histidine (His) tag shown in FIG. 4A that is bound with the fusion protein. Alternatively, these markers may include various other identifiers, such as various affinity tags, fluorescent labels, isotopes, radioactive tags, colorimetric or luminescent tags, as known to those skilled in the art.

It is further contemplated that the protein PE38 may provide the active, toxic moiety for various antibody constructs contemplated by the current invention. These constructs are selective for the Fn14 receptor expressed (over-expressed) on particular cells, such as any of the cells described above. The antibody constructs may include various other synergistic agents, for example, catalysts such as liposomes, penetrating agents, additives to maintain physiological and/or istonic stability, buffers, preservatives, other epitopes with alternative binding affinities for alternative receptor sites on a cell, and the like. It is contemplated that such secondary or synergistic agents may be included through the use of standard techniques and methodologies for producing such a complex as are well-known to those of ordinary skill in the art. The antibody construct may take various forms, for example, an immunotoxin construct that includes the active toxic moiety of the current invention fused with an anti-Fn14 antibody. Alternatively, the active toxic moiety of the current invention may be used and conjugatively associated or bound with an anti-Fn14 antibody. Thus, the antibody constructs of the current invention contemplate the replacement of the TWEAK protein and its replacement with an antibody sequence that provides for selective binding with the surface of a cell at the Fn14 receptor binding region. It is further contemplated that antibody/toxic moiety constructs may include the TWEAK protein as a secondary component feature and may be used as a secondary binding agent for assisting with the or further promoting the binding of the antibody/toxic moiety complex to the surface of a cell.

It is well known in the art that various secondary component features, such as those described above, which provide an advantageous particular characteristic or numerous characteristics, may be included in any of the antibody constructs contemplated by the current invention. Thus, the antibodies of the current invention may use and/or deliver the cytotoxic effect for and/or to a particular cell in a manner similar to that provided by the TWEAK-PE38 fusion protein. The use of standard techniques and methodologies for producing the antibody construct(s) are contemplated by the current invention and are well-known by those of ordinary skill in the art.

The production of antibodies that are selective for the Fn14 receptor extracellular domain may occur through the use of various processes and techniques that are well-known to those of ordinary skill in the art. It is further contemplated that antibody fragments may be produced and used to provide the selective binding affinity for the Fn14 receptor and delivery of the toxic moiety of the current invention. It is further understood that antibodies may be created, using the standard and well-known techniques and methodologies to those skilled in the art, from the known TWEAK receptor binding region being expressed on the surface of particular cells. For example, from this known sequence one or more selectively binding or high affinity binding sequences may be identified. These sequences may be used to form various types of antibody constructs which include the toxic moiety of the current invention. Thus, whether the construct takes the immunotoxin or conjugative form, as stated previously, the production of antibodies including the novel toxic moiety structure of the current invention are contemplated to fall within the scope and spirit of the current invention.

In general, delivery mechanisms for the fusion protein of the current invention, either alone or in combination with other compounds and/or structures, may range from ingestion, such as various solid or liquid compounds, to parenteral, such as injection (intravenous, subcutaneous, intratumoral, intraperitoneal or intramuscular) and suppositories (rectal or vaginal), to topical, such as emulsions, creams, lotions, gels, and the like. The delivery mechanisms may promote the efficient transportation of the fusion protein to the location of the diseased cell, the efficient binding of the fusion protein with the receptor, the efficient internalization of the fusion protein and/or toxic moiety, and the efficient movement of the fusion protein/toxic moiety within the cytosolic compartment.

The TWEAK-PE38 fusion protein may be formulated into various therapeutics for various types of cells, such as the diseased cells mentioned above. The therapeutic may simply include the fusion protein of the current invention itself or it may present the fusion protein in combination with various other active/non-active secondary components. Secondary components may include various reagents, excipients, diluents, buffers, catalysts, additives, preservatives, solution(s), liposomes, and the like, and may be included in the therapeutic construct through use of the various techniques and methodologies known to those skilled in this art field. In a preferred embodiment, the TWEAK-PE fusion protein provides a cancer therapeutic. The therapeutic may be of a general nature, wherein the fusion protein is non-specific to the type or form of cancer and binds with all malignant or benign cells expressing a particular receptor site. Alternatively, the therapeutic may be capable of binding specifically with particular cancerous cell types. This capability may be promoted by the binding of the fusion protein of the current invention with various other compounds, active moieties, and the like, that may be specific for a cancer type.

Formulations may range from aqueous solutions (liquid drink, suspensions, solutions), to organic compounds (granules, tablets, capsules, emulsions), and the like. The formulations (e.g., therapeutic) may also include other secondary or synergistic agents such as those mentioned above and other various agents, such as penetrating agents, which may be employed to increase the bio-availability of the moiety across a membrane. It is contemplated that the production of these formulations may occur through the use of standard techniques and methodologies that are well-known to those skill in the art.

Administration of a therapeutic including the fusion protein of the current invention may be accomplished through the use of any of the various delivery mechanisms identified above. For example, the TWEAK-PE fusion protein may be the active moiety within a vaccine formulation or simply one of several active moieties. The vaccine formulation may include a pharmacologically effective amount of the fusion protein active moiety. The administration may occur in combination with a physiologically-acceptable, non-toxic, liquid carrier, compatible with the fusion protein. Suitable carriers may include physiological saline, phosphate-buffered saline, Tris (hydroxymethyl aminomethane (TRIS)), Tris-buffered saline, and the like. Adjuvants may be included to enhance immune response. For example, aluminum hydroxide, aluminum phosphate, liposomes, Freund's Incomplete Adjuvant (FCA), and the like. Additives, such as various buffers and preservatives which may assist in maintaining isotonicity, physiological pH, stability, sterility, and the like, may also be included. Formulations may include various emulsifying and/or suspending agents, pharmaceutically acceptable diluents, and the like, to control delivery and dose amount of the vaccine.

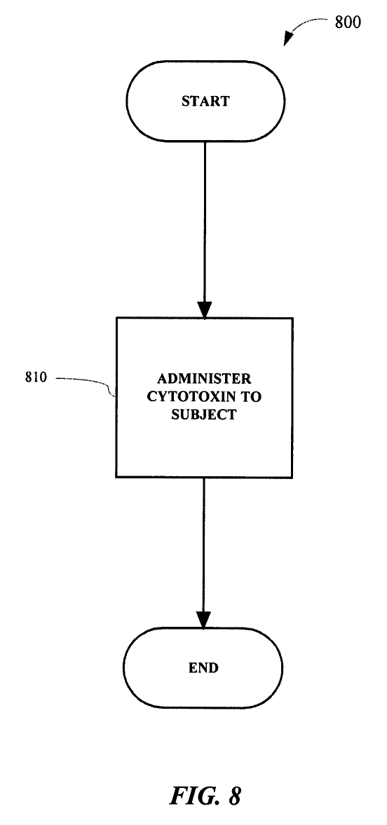

The current invention may also be used for the treatment of subjects that are in need, such as those afflicted with and suffering from the various cancers, other diseases, and/or cellular insult response processes, as previously described. The subjects may be individual animals, wherein any and all types of animals, including mammals, such as humans, and the like, are subjects contemplated by the current invention. In a preferred embodiment of the current invention, a treatment method 800 is shown in FIG. 8 to include a single step 810 of administering to the subject a cytotoxin including the fusion protein of the current invention, whereby the fusion protein will bind with the cell. As described above, the result of the binding of the fusion protein with the diseased cell is the death of that cell and therefore the disease is prevented from further growth. As stated above, the formulation and form of the fusion protein may vary to accommodate the needs of a manufacturer, health care provider and/or afflicted subject. The amount given shall preferably be a pharmacologically effective amount to cause the death of those cells that are infected by the disease and therefore are promoting its growth without also generating a negative response, such as the destruction of healthy cells.

The delivery of the fusion protein may be into the bloodstream for travel to or contact with the diseased cell(s). It is also contemplated that the delivery of the fusion protein may be directly into a tumor, such as a solid cancer tumor (malignant or benign). This solid tumor delivery may require different formulation(s) than those required for a more systemic approach and are contemplated by the current invention. For example, the solid tumor therapeutics may require additional membrane transport mechanisms (e.g., liposomes) be loaded with the fusion protein in order to promote travel across intercellular and intracellular spaces and attachment to the cell surface receptor.

It is contemplated that the current method include the step of administering the therapeutic in all of the various formulations and delivery forms as have been previously described. The treatment method may include the combination of the fusion protein of the current invention and other therapeutics, which may address various other cell conditions. The combination may also enhance or increase the lethality of the therapeutic for the disease cell being targeted. The combination may be deployed in the form of a single therapeutic or the combination of multiple therapeutics. Delivery may be in a single stage or multi-stage procedure or require other conditions be met for effectiveness to be realized.

### Study Method

### (a) Design

1) Fn14 mRNA expression levels are elevated in certain cancer specimens and in glioma cells actively invading the brain parenchyma (39). In agreement with these findings, immunohistochemical staining experiments have indicated that Fn14 protein expression is very low in normal brain cells but significantly higher in GBM core and invasive rim cells. Thus, Fn14 is an attractive glioma cell surface marker because it is overexpressed by both proliferating tumor core cells and invading tumor rim cells.

It is contemplated that the chimeric TWEAK-PE38 fusion protein has cytotoxic activity on Fn14-positive cells proving effective for targeting both the stationary and invasive glioma cell subpopulations in various environments. The TWEAK fusion protein may be internalized by receptor-mediated endocytosis allowing the toxin moiety to enter the cytosolic compartment in order to ADP ribosylate EF-2 and promote cell death (6). It has been reported that several TNF superfamily members undergo internalization following binding to their specific cell surface receptors (20-24). The current invention indicates that TWEAK is also efficiently internalized by Fn14-positive cells (FIGS. 2 and 3).

Although other fusion protein toxins targeted to glioma cell surface receptors have shown some efficacy in human clinical trials, TWEAK-PE may be a more selective and a safer agent. For example, the IL-13-PE38QQR fusion protein toxin presently in Phase III clinical trials (the PRECISE trial; Neopharm Inc.) binds two cell surface receptors, IL-13Rα1 and IL-13Rα2 (25), only one of which is overexpressed on glioma cells (IL-13Rα2) (26). Furthermore, since IL-13Rα2 has been found in soluble form in vivo (27), the fusion protein toxin may also bind a circulating molecule. In contrast, TWEAK binds with high affinity to one small membrane-anchored receptor, Fn14.

2) Construction of bacterial expression plasmids and cell transformation: As indicated previously, two different TWEAK-PE38 fusion proteins may be produced (FIG. 4). The N-terminal region of these proteins will consist of either the TWEAK extracellular, receptor-binding domain (149 aa in length; aa 101-249, (SEQ ID NO. 4), (35)) or a PE mutant called PE38 (445 aa in length). PE is a single-chain bacterial protein with three major domains: an N-terminal cell-binding domain (aa 1-252), an amphipathic helix-containing translocation domain containing a critical furin cleavage site (aa 253-364), and a C-terminal ADP-ribosylation domain (aa 396-613). The PE38 protein is missing the entire cell-binding domain and domain 1b (aa 365-395) (6-7). The pET Procaryotic Expression System (Novagen) may be used. There are many different plasmids and host strains that can be utilized in this system. As a starting point, the plasmid pET45 (T7 promoter-based; IPTG-inducible; ampicillin resistance marker) and the genetically-engineered E. coli strain BL21(DE3)pLysS may be used. This plasmid and host strain were previously successfully utilized for high-yield production of biologically active IL-13-PE38 fusion protein (31). In a preferred embodiment, the plasmid and competent cells are obtained from Novagen. DNA fragments encoding the TWEAK receptor-binding domain and the PE38 protein may be cloned into pET45 using PCR-based cloning strategies and appropriate restriction sites. The constructs may be verified by DNA sequence analysis. Transformation through use of bacterial (or insect) cells may be performed as described (31).

The TWEAK-PE38 fusion proteins may also be fused to an IZ domain (33 aa in length, (SEQ ID NO. 3), (30)). Thus, a fusion protein containing an N-terminal IZ domain may be produced and DNA fragments encoding the IZ domain may be cloned into pET45 in a similar manner as that described above. It has been shown that soluble TNF superfamily ligand monomers, including TWEAK (28), spontaneously associate into a homotrimeric structure that binds with high affinity to the appropriate cell surface receptor(s). Nevertheless, it has been demonstrated that at least two members of the TNF superfamily, CD40L (29) and TRAIL (30), may have increased biological activity and stability when they are engineered to contain an N-terminal isoleucine zipper (IZ) trimerization domain. Also described is a chimeric soluble Fas (CD95) ligand (FasL) fused to an IZ motif for self-oligomerization (38).

3) Purification of fusion proteins from bacterial cells: There are two very detailed PE38 fusion protein expression and purification protocols in the literature (31-32). These protocols describe all of the steps involved, including IPTG induction, cell harvesting and lysis, inclusion body enrichment and washing, denaturation and refolding of the protein, dialysis, and chromatography (ion exchange and gel filtration). The expression and purification of the fusion proteins shown in FIGS. 4A, 4B, and 4C may be accomplished utilizing the protocols outlined above. It is contemplated that modifications may have to be made in order to express and purify the three proteins. Detection of the fusion proteins may be accomplished at the various purification steps by performing SDS-PAGE and protein staining experiments, purification of the TWEAK-PE38 fusion proteins, and the PE38 protein itself, which has been achieved using a nickel-agarose affinity resin (FIG. 4B). If necessary, Western blot analysis may be employed using anti-TWEAK (Cell Sciences) and anti-PE (Sigma) polyclonal antibodies.

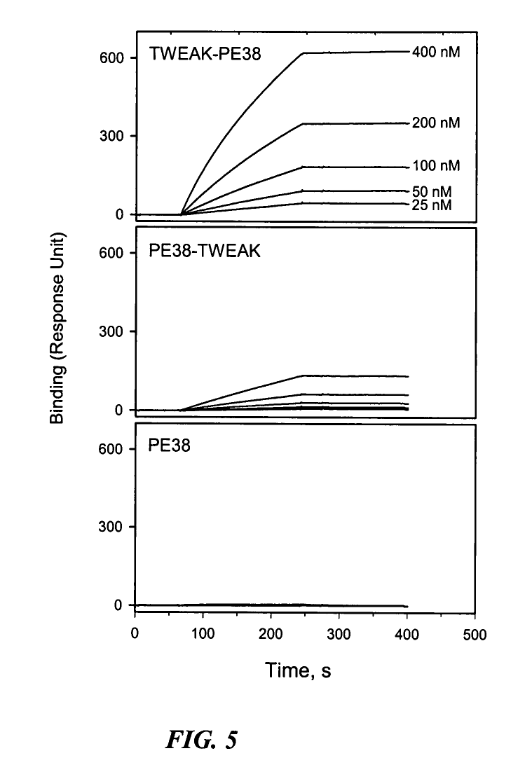

4) Fn14-binding assays: Binding of the purified TWEAK-PE38 and PE38-TWEAK proteins to the Fn14 receptor may be detected by performing surface plasmon resonance experiments using various methodologies, such as employing the BIAcore 3000 instrument. Detection of these proteins has been accomplished using this assay, as shown in FIG. 5.

5) Quantitative cytotoxicity assays using Fn14-positive and -negative cell lines cultured in vitro:

a. Cells: a panel of different cell lines may be used to determine cytotoxcity of the fusion proteins of the present invention. Fn14-positive human tumor cells used may include T98G and SF767, as well as Fn14-positive human endothelial cells (3). The LoVo colon carcinoma cell line may be used in the proposed assays because they may function as an ideal negative control cell line. FACS analysis indicates that these cells express Fn14 cell surface receptors (data not shown); however, they also express a catalytically inactive form of furin (33). Since furin is the intracellular protease that cleaves PE domain 2, a step that is absolutely required for translocation of the toxin into the cytosol (34), then the TWEAK-PE38 and PE38-TWEAK proteins may bind these cells and may have no effect on protein synthesis (see below). In regard to Fn14-negative human cells, to date only one cell line, the Jurkat T cell leukemia line, has been reported to be Fn14-negative (as assayed by FACS analysis) (35).

b. Protein synthesis inhibition assays: Protein synthesis levels (as measured by amino acid incorporation into newly synthesized proteins) directly correlate with the number of viable cells in a tissue culture well (36); therefore, the cytotoxic activity of the fusion proteins may be determined using a protein synthesis assay. The various cell lines may be plated in triplicate in leucine-free medium at 10⁴ cells/well (96-well flat-bottom microtiter plates) in the absence or presence of various concentrations (0.1 to 1000 ng/ml) of each fusion protein. The cells may be incubated at 37° C., 5% CO₂ for 24 hr and then ³H-leucine (NEN; final concentration 20 μCi/ml) may be added. Cells may be incubated for an additional 4 hr and then a cell harvester (Packard) used to process the samples. The amount of ³H-leucine incorporation into cells may be measured using a scintillation counter. The IC50 value (defined as the concentration of toxin that inhibits leucine incorporation by 50% compared with control, untreated wells) for each fusion protein calculated using GraphPad Prism software.

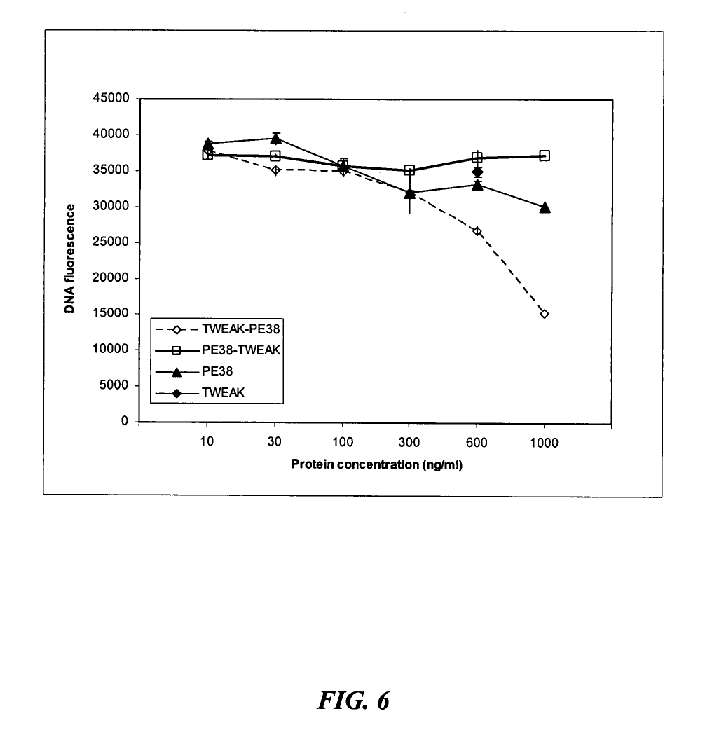

c. Cell growth inhibition assays: The cytotoxic activity of the fusion proteins may also be tested using cell proliferation assays, for example the MTT or CyQuant assays available from Invitrogen. Preliminary experiments using the latter assay have indicated that the TWEAK-PE38 fusion protein has cytotoxic activity when added to breast cancer cells (FIG. 6).

Blocking experiments to evaluate the specificity of the TWEAK-PE38 and PE38-TWEAK cytotoxic effect may be performed. For example, the cytotoxic activity should be blocked by co-incubation with an excess (10-100×) of recombinant soluble TWEAK. Also, pre-incubation (30 min, 37° C.) of the two fusion proteins with either an anti-TWEAK neutralizing antibody (CARL-1; eBioscience) or an Fn14-Fc decoy receptor (made in our laboratory, see 3, 19) should block the cytotoxic effect, providing that these two reagents can still recognize the TWEAK region of the fusion proteins. Several different concentrations of these blocking reagents, and their appropriate negative controls (normal mouse IgG3 (eBioscience) and mouse Fc protein (made in our laboratory (3, 19), respectively), may be used in this series of ³H-leucine incorporation experiments.

6) Qualitative cytotoxicity assays using murine brain slices: To further investigate the targeting specificity of the TWEAK fusion proteins, GFP-tagged, Fn14-positive glioma cells may be placed onto brain slices and fusion proteins added to the culture medium. Briefly, age- and sex-matched C57/BL6:WT and C57/BL6:Fn14-KO mice may be euthanized and brain slices prepared as described earlier. GFP-tagged glioma cells (10⁵) may be gently placed in a pinhole created with a micropipette. The recombinant fusion proteins may be immediately added to the top culture medium of the Transwell chambers at several concentrations (as determined above). Human glioma cell and mouse brain slice cell viability may be monitored as follows. Propidium iodide (PI; a red-fluorescent, cell impermeant dye; purchased from Molecular Probes) may be added to the cultures (25 μg/ml, 15 min, room temperature) at several different time points (e.g., 6, 12, 18, 24 hr) and then the slices may be examined using an inverted fluorescent microscope (no cell fixation). Human and mouse cell death will be accompanied by loss of plasma membrane integrity. Thus, as the glioma cells die, GFP may be released into the culture medium and PI may diffuse into the cell and bind cellular DNA. Consequently, these cells may become GFP-negative and PI-positive. If any brain slice cells die, they may become PI-positive. The human glioma cells may be killed regardless of the brain slice genotype. The Fn14-WT brain slice cells may or may not be killed, depending on their level of Fn14 expression; however, Fn14-KO brain slice cells may be completely resistant to TWEAK-PE38 exposure.

It is contemplated that the current invention may employ various protocol optimization experiments to promote the expression and or purification of the fusion proteins. Further, the contemplated use of the pET Procaryotic Expression System (Novagen), may be substituted with other systems that may allow the production of biologically active protein; for example, the SUMOpro Protein Expression and Purification System (Lifesensors Inc.). In this system, the protein of interest is expressed with an N-terminal SUMO (small ubiquitin-like modifier) fusion tag, and this modification has been shown to enhance protein expression levels, facilitate purification, and promote solubility and correct folding. At the end of the purification protocol, the SUMO tag is released from the target protein using SUMO Protease. Both the tag and the protease are then isolated away from the target protein by Ni-NTA affinity chromatography. The TWEAK-PE38 fusion proteins may also be expressed in insect cells and then purified.

In the exemplary embodiments, the method(s) or process(es) disclosed may have been presented as a sequence of steps having a particular order. Any of the particular order of steps presented are not intended to limit and should not be construed as limitations of the current invention to any particular order wherein the method(s) or process(es) themselves are not dependent upon the execution of the steps in any particular order. It will be understood by those of ordinary skill in the art that alternative sequences of the steps, alternative steps, fewer steps, and additional steps may be employed without departing from the scope and spirit of the present invention. Further, the claims herein presented below, which provide a particular sequence of steps in an order should not be limited to their particular order as those of skill in the art will appreciate that various sequences may be employed without departing from the scope and spirit of the present invention.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

### REFERENCES

The following references have been cited within the specification of the instant application and are herein incorporated by reference in their entireties:
1. Rich, J. N. and Bigner, D. D. Development of novel targeted therapies in the treatment of malignant glioma. Nat. Rev. Drug Discov. 3: 430-446, 2004.
2. Lesniak, M. S. and Brem, H. Targeted therapy for brain tumours. Nat. Rev. Drug Discov. 3: 499-508, 2004.
3. Donohue, P. J., Richards, C. M., Brown, S. A., Hanscom, H. N., Buschman, J., Thangada, S., Hla, T., Williams, M. S., and Winkles, J. A. TWEAK is an endothelial cell growth and chemotactic factor that also potentiates FGF-2 and VEGF-A mitogenic activity. Arterioscler. Thromb. Vasc. Biol. 23: 594-600, 2003.
4. Nabors, L. B. Targeted molecular therapy for malignant gliomas. Curr. Treat. Options Oncol. 5: 519-526, 2004.
5. Mischel, P. S. and Cloughesy, T. F. Targeted molecular therapy of GBM. Brain Pathol. 13: 52-61, 2003.
6. Frankel, A. E., Kreitman, R. J., and Sausville, E. A. Targeted toxins. Clin. Cancer Res. 6: 326-334, 2000.
7. Kreitman, R. J. Recombinant toxins for the treatment of cancer. Curr. Opin. Mol. Ther. 5: 44-51, 2003.
8. Frankel, A. E., Powell, B. L., Vallera, D. A., and Neville, D. M., Jr. Chimeric fusion proteins—diphtheria toxin-based. Curr. Opin. Investig. Drugs 2: 1294-1301, 2001.
9. Weaver, M. and Laske, D. W. Transferrin receptor ligand-targeted toxin conjugate (Tf-CRM107) for therapy of malignant gliomas. J. Neurooncol. 65: 3-13, 2003.
10. Rustamzadeh, E., Li, C., Doumbia, S., Hall, W. A., and Vallera, D. A. Targeting the over-expressed urokinase-type plasminogen activator receptor on glioblastoma multiforme. J. Neurooncol. 65: 63-75, 2003.
11. Liu, T. F., Cohen, K. A., Ramage, J. G., Willingham, M. C., Thorburn, A. M., and Frankel, A. E. A diphtheria toxin-epidermal growth factor fusion protein is cytotoxic to human glioblastoma multiforme cells. Cancer Res. 63: 1834-1837, 2003.
12. Sampson, J. H., Akabani, G., Archer, G. E., Bigner, D. D., Berger, M. S., Friedman, A. H., Friedman, H. S., Herndon, J. E., Kunwar, S., Marcus, S., McLendon, R. E., Paolino, A., Penne, K., Provenzale, J., Quinn, J., Reardon, D. A., Rich, J., Stenzel, T., Tourt-Uhlig, S., Wikstrand, C., Wong, T., Williams, R., Yuan, F., Zalutsky, M. R., and Pastan, I. Progress report of a Phase I study of the intracerebral microinfusion of a recombinant chimeric protein composed of transforming growth factor (TGF)-alpha and a mutated form of the Pseudomonas exotoxin termed PE-38 (TP-38) for the treatment of malignant brain tumors. J. Neurooncol. 65: 27-35, 2003.
13. Kawakami, M., Kawakami, K., and Puri, R. K. Interleukin-4-Pseudomonas exotoxin chimeric fusion protein for malignant glioma therapy. J. Neurooncol. 65: 15-25, 2003.
14. Puri, R. K., Hoon, D. S., Leland, P., Snoy, P., Rand, R. W., Pastan, I., and Kreitman, R. J. Preclinical development of a recombinant toxin containing circularly permuted interleukin 4 and truncated Pseudomonas exotoxin for therapy of malignant astrocytoma. Cancer Res. 56: 5631-5637, 1996.
15. Debinski, W., Obiri, N. I., Powers, S. K., Pastan, I., and Puri, R. K. Human glioma cells overexpress receptors for interleukin 13 and are extremely sensitive to a novel chimeric protein composed of interleukin 13 and Pseudomonas exotoxin. Clin. Cancer Res. 1: 1253-1258, 1995.
16. Husain, S. R. and Puri, R. K. Interleukin-13 receptor-directed cytotoxin for malignant glioma therapy: from bench to bedside. J. Neurooncol. 65: 37-48, 2003.
17. Li, C., Hall, W. A., Jin, N., Todhunter, D. A., Panoskaltsis-Mortari, A., and Vallera, D. A. Targeting glioblastoma multiforme with an IL-13/diphtheria toxin fusion protein in vitro and in vivo in nude mice. Protein Eng. 15: 419-427, 2002.
18. Weber, F., Asher, A., Bucholz, R., Berger, M., Prados, M., Chang, S., Bruce, J., Hall, W., Rainov, N. G., Westphal, M., Warnick, R. E., Rand, R. W., Floeth, F., Rommel, F., Pan, H., Hingorani, V. N., and Puri, R. K. Safety, tolerability, and tumor response of IL4-Pseudomonas exotoxin (NBI-3001) in patients with recurrent malignant glioma. J. Neurooncol. 64: 125-137, 2003.
19. Yepes, M., Brown, S. A., Moore, E. G., Smith, E. P., Lawrence, D. A., and Winkles, J. A. A soluble Fn14-Fc decoy receptor reduces infarct volume in a murine model of cerebral ischemia. Am. J. Pathol. 166: 511-520, 2005.
20. Gullberg, U., Lantz, M., Nilsson, E., and Olsson, I. Characterization of the receptor for lymphotoxin; a spontaneous internalization without recycling and ligand-induced downregulation in HL-60 cells. Eur. J. Cell Biol. 49: 334-340, 1989.
21. Pennica, D., Lam, V. T., Mize, N. K., Weber, R. F., Lewis, M., Fendly, B. M., Lipari, M. T., and Goeddel, D. V. Biochemical properties of the 75-kDa tumor necrosis factor receptor. Characterization of ligand binding, internalization, and receptor phosphorylation. J. Biol. Chem. 267: 21172-21178, 1992.
22. Schutze, S., Machleidt, T., Adam, D., Schwandner, R., Wiegmann, K., Kruse, M. L., Heinrich, M., Wickel, M., and Kronke, M. Inhibition of receptor internalization by monodansylcadaverine selectively blocks p55 tumor necrosis factor receptor death domain signaling. J. Biol. Chem. 274: 10203-10212, 1999.
23. Zhang, X. D., Franco, A. V., Nguyen, T., Gray, C. P., and Hersey, P. Differential localization and regulation of death and decoy receptors for TNF-related apoptosis-inducing ligand (TRAIL) in human melanoma cells. J. Immunol. 164: 3961-3970, 2000.
24. Manning, E., Pullen, S. S., Souza, D. J., Kehry, M., and Noelle, R. J. Cellular responses to murine CD40 in a mouse B cell line may be TRAF dependent or independent. Eur. J. Immunol. 32: 39-49, 2002.
25. Hershey, G. K. IL-13 receptors and signaling pathways: an evolving web. J. Allergy Clin. Immunol. 111: 677-690, 2003.
26. Joshi, B. H., Plautz, G. E., and Puri, R. K. Interleukin-13 receptor alpha chain: a novel tumor-associated transmembrane protein in primary explants of human malignant gliomas. Cancer Res. 60: 1168-1172, 2000.
27. Zhang, J. G., Hilton, D. J., Willson, T. A., McFarlane, C., Roberts, B. A., Moritz, R. L., Simpson, R. J., Alexander, W. S., Metcalf, D., and Nicola, N. A. Identification, purification, and characterization of a soluble interleukin (IL)-13-binding protein. Evidence that it is distinct from the cloned Il-13 receptor and Il-4 receptor alpha-chains. J. Biol. Chem. 272: 9474-9480, 1997.
28. Schneider, P., Schwenzer, R., Haas, E., Muhlenbeck, F., Schubert, G., Scheurich, P., Tschopp, J., and Wajant, H. TWEAK can induce cell death via endogenous TNF and TNF receptor 1. Eur. J. Immunol. 29: 1785-1792, 1999.
29. Morris, A. E., Remmele, R. L., Jr., Klinke, R., Macduff, B. M., Fanslow, W. C., and Armitage, R. J. Incorporation of an isoleucine zipper motif enhances the biological activity of soluble CD40L (CD154). J. Biol. Chem. 274: 418-423, 1999.
30. Kim, M. H., Billiar, T. R., and Seol, D. W. The secretable form of trimeric TRAIL, a potent inducer of apoptosis. Biochem. Biophys. Res. Commun. 321: 930-935, 2004.
31. Joshi, B. H. and Puri, R. K. Optimization of expression and purification of two biologically active chimeric fusion proteins that consist of human interleukin-13 and Pseudomonas exotoxin in Escherichia coli. Protein Expr. Purif. 39: 189-198, 2005.
32. Pastan, I., Beers, R., and Bera, T. K. Recombinant immunotoxins in the treatment of cancer. Methods Mol. Biol. 248: 503-518, 2004.
33. Takahashi, S., Nakagawa, T., Kasai, K., Banno, T., Duguay, S. J., Van de Ven, W. J., Murakami, K., and Nakayama, K. A second mutant allele of furin in the processing-incompetent cell line, LoVo: evidence for involvement of the homo B domain in autocatalytic activation. J. Biol. Chem. 270: 26565-26569, 1995.
34. Inocencio, N. M., Moehring, J. M., and Moehring, T. J. Furin activates Pseudomonas exotoxin A by specific cleavage in vivo and in vitro. J. Biol. Chem. 269: 31831-31835, 1994.
35. Nakayama, M., Ishidoh, K., Kojima, Y., Harada, N., Kominami, E., Okumura, K., and Yagita, H. Fibroblast growth factor-inducible 14 mediates multiple pathways of TWEAK-induced cell death. J. Immunol. 170: 341-348, 2003.
36. Puri, R. K., Leland, P., Obiri, N. I., Husain, S. R., Mule, J., Pastan, I., and Kreitman, R. J. An improved circularly permuted interleukin 4-toxin is highly cytotoxic to human renal cell carcinoma cells: introduction of gamma c chain in RCC cells does not improve sensitivity. Cell Immunol. 171: 80-86, 1996.
37. Tanabe, K., I. Bonilla, J. A. Winkles and S. M. Strittmatter: Fibroblast growth factor-inducible-14 is induced in axotomized neurons and promotes neurite outgrowth. J Neurosci 23, 9675-9686 (2003) 38. Tetsuya Shiraishia, *, Kenji Suzuyamaa, Hiroaki Okamotoa, Toshihiro Minetaa, Kazuo Tabuchia, Kazuyuki Nakayamab, Yusuke Shimizub, Junko Tohmab, Takuo Ogiharab, Hiroyasu Nabab, Hidenori Mochizukib, Shigekazu Nagatac: Increased cytotoxicity of soluble Fas ligand by fusing isoleucine zipper motif. BBRC 322, (2004) 197-202.
39. Winkles J. A., Tran, N. L., Brown, S. A. N., Stairs, N., Lundciffe, H. E., and Berens, M. E.: Role of TWEAK and Fn14 in tumor Biology. Frontiers in Biosciences, 2007, 12, p. 2761-2771.

What is claimed is:

1. A cytotoxin, comprising:
a tumor necrosis factor like weak inducer of apoptosis (TWEAK) receptor-binding domain fused with a Pseudomonas exotoxin (PE38),
wherein the TWEAK receptor-binding domain binds with a cell.

2. The cytotoxin of claim 1, wherein the binding of the TWEAK receptor-binding domain with the cell occurs via an expressed cell surface receptor.

3. The cytotoxin of claim 2, wherein the cell surface receptor is a fibroblast growth factor-inducible 14 (Fn14) receptor.

4. The cytotoxin of claim 3, wherein the Fn14 receptor is over-expressed on the cell surface as compared to a normal cell.

5. The cytotoxin of claim 1, wherein the TWEAK receptor-binding domain is at least one of the N-terminus or C-terminus of the cytotoxin.

6. The cytotoxin of claim 1, wherein the TWEAK receptor-binding domain sequence of amino acids may be at least one of a range of 8 to 297 amino acids in length or 147 amino acids in length.

7. The cytotoxin of claim 1, wherein the sequence of PE38 includes amino acids 253 to 364 and 396 to 613 of a Pseudomonas exotoxin (PE).

8. The cytotoxin of claim 1, wherein the cell is selected from the group consisting of a cancer cell, wherein the cancer cell may be selected from the group consisting of liver, brain, breast, colon, cervical, testicular, and esophageal cancer, a diseased cell, wherein the diseased cells may be selected from the group consisting of atherosclerosis, stroke, lupus, nephritis, multiple sclerosis, and rheumatoid arthritis, and a cell undergoing at least one of a regenerative or inflammation response.

9. The cytotoxin of claim 1, wherein the cytotoxin further includes at least one of an isoleucine zipper (IZ) domain fused to at least one of the N-terminus or C-terminus of the TWEAK receptor-binding domain or a glycine/serine linker which separates the TWEAK receptor-binding domain and PE38 proteins.

10. A cytotoxin, comprising:
an amino terminus, the amino terminus including a tumor necrosis factor like weak inducer of apoptosis (TWEAK) receptor-binding domain, fused with a Pseudomonas exotoxin (PE38),
wherein the TWEAK receptor-binding domain binds with a cell.

11. The cytotoxin of claim 10, wherein the TWEAK receptor-binding domain sequence of amino acids may be at least one of a range of 8 to 297 amino acids in length or 147 amino acids in length.

12. The cytotoxin of claim 10, wherein the sequence of PE38 includes amino acids 253 to 364 and 396 to 613 of a Pseudomonas exotoxin (PE).

13. The cytotoxin of claim 10, wherein the cell is selected from the group consisting of a cancer cell, wherein the cancer cell may be selected from the group consisting of liver, brain, breast, colon, cervical, testicular, and esophageal cancer, a diseased cell, wherein the diseased cells may be selected from the group consisting of atherosclerosis, stroke, lupus, nephritis, multiple sclerosis, and rheumatoid arthritis, and a cell undergoing at least one of a regenerative or inflammation response.

14. The cytotoxin of claim 10, wherein the cell is bound at a cell surface receptor

15. The cytotoxin of claim 14, wherein the cell surface receptor is a fibroblast growth factor-inducible 14 (Fn14) receptor.

16. The cytotoxin of claim 15, wherein the Fn14 receptor is over-expressed on the cell surface as compared to a normal cell.

17. The cytotoxin of claim 10, wherein the cytotoxin further includes at least one of an isoleucine zipper (IZ) domain fused to at least one of the N-terminus or C-terminus of the TWEAK receptor-binding domain or a glycine/serine linker which separates the TWEAK receptor-binding domain and PE38 proteins.

18. A method of inducing death of a cell, comprising:
contacting the cell with a cytotoxin, the cytotoxin including a tumor necrosis factor like weak inducer of apoptosis (TWEAK) receptor binding domain fused with a mutant Pseudomonas exotoxin (PE38), that binds the TWEAK receptor-binding domain with a cell, inducing cell death.

19. The method of claim 18, wherein the inducing cell death step is a result of internalizing the PE38 into a cells cytosolic compartment.

20. The method of claim 18, wherein contacting results in the binding of at least one of the N-terminus or C-terminus of the cytotoxin with the cell.

21. The method of claim 18, wherein the contacting results in the binding of the cytotoxin with a fibroblast growth factor-inducible 14 (Fn14) receptor.

22. The method of claim 18, wherein the cell is selected from the group consisting of a cancer cell, wherein the cancer cell may be selected from the group consisting of liver, brain, breast, colon, cervical, testicular, and esophageal cancer, a diseased cell, wherein the diseased cells may be selected from the group consisting of atherosclerosis, stroke, lupus, nephritis, multiple sclerosis, and rheumatoid arthritis, and a cell undergoing at least one of a regenerative or inflammation response.

23. A method of treating a subject in need thereof, comprising:
administering a cytotoxin, the cytotoxin including a tumor necrosis factor like weak inducer of apoptosis (TWEAK) receptor-binding domain fused with a Pseudomonas exotoxin (PE38), that binds the TWEAK receptor-binding domain with a cell.

24. The method of claim 23, wherein the administration of the cytotoxin is of a pharmacologically effective amount of the cytotoxin to effect the binding of at least one of the N-terminus or C-terminus of the cytotoxin with the cell.

25. The method of claim 23, further comprising the step of formulating the cytotoxin in combination with at least one of a catalyst, chaperone, assembly or transport biomolecule, liposome, reagent, excipient, diluent, emulsifier, penetrating agent, additive, preservative, buffer, solution, saline solution, tris, or organic compound.

26. The method of claim 23, wherein the formulation step provides the cytotoxin in at least one of an ingestible, parenteral, or topical formulation that is administered to the subject.

27. The method of claim 23, further comprising the step of inducing cell death as a result of internalizing the PE38 into a cytosolic compartment of the cell, wherein the cell is selected from the group consisting of a cancer cell, wherein the cancer cell may be selected from the group consisting of liver, brain, breast, colon, cervical, testicular, and esophageal cancer, a diseased cell, wherein the diseased cells may be selected from the group consisting of atherosclerosis, stroke, lupus, nephritis, multiple sclerosis, and rheumatoid arthritis, and a cell undergoing at least one of a regenerative or inflammation response.

28. A cytotoxin, comprising:
an anti-Fn14 antibody associated with a Pseudomonas exotoxin (PE38),
wherein the anti-Fn14 antibody binds with a cell.

29. The cytotoxin of claim 28, wherein the association occurs through at least one of a fusion or conjugation of the anti-Fn14 antibody with the PE38.

30. The cytotoxin of claim 28, wherein the anti-Fn14 antibody binds with a fibroblast growth factor-inducible 14 (Fn14) receptor.
